(19) United States
(12) Patent Application Publication
(10) Pub. No.: US 2008/0066746 A1
(43) Pub. Date: Mar. 20, 2008

(54) Method and device for placing an endotracheal tube

(76) Inventors: Lindsey A. Nelson, New Richmond, OH (US); Steven J. Lisco, Cincinnati, OH (US)

Correspondence Address: Mark F. Smith, 905 Ohio Pike, Cincinnati, OH, 45245, US

(21) Appl. No.: 11/900,797

(22) Filed: Sep. 13, 2007

Related U.S. Application Data
(60) Provisional application No. 60/844,429, filed on Sep. 14, 2006.

(51) Int. Cl. A61M 16/00

(52) U.S. Cl. 128/200.26

(57) ABSTRACT

Device and method for inserting an endotracheal tube or for replacing an endotracheal tube that already exists in the trachea with a new endotracheal tube. The device comprises a tubular structure effective for providing ventilation to the patient during replacement of the endotracheal tube. A stylet is provided having a taper section that gradually increases in diameter from the distal end thereby eliminating the difference in diameters between the tubular structure and the endotracheal tube and for facilitating entry of the new endotracheal tube into the trachea of the patient.

### CROSS-REFERENCE TO RELATED APPLICATION

This application claims benefit of U.S. Provisional Application Ser. No. 60/844,429, filed Sep. 14, 2006, which is directed to a method and apparatus for replacing a placed endotracheal tube.

### TECHNICAL FIELD

This invention relates to medical devices and more particularly to a method and apparatus for placing an endotracheal tube in a patient and more particularly, to a method and apparatus for placing an endotracheal tube in a patient or for exchanging an existing inserted endotracheal tube (ETT) and placing a new ETT by another conduit such as a fiberoptic bronchoscope.

### BACKGROUND OF THE INVENTION

The placement of an ETT into a patient often requires the assistance of a guide. In situations where the ETT requires exchange, a tubular obturator is typically used as a guide; the obturator is passed down the existing inserted ETT, the ETT is then removed and a new ETT is passed over the obturator in attempt to guide its placement into the patient's trachea. A fiberoptic bronchoscope is also typically used as a guide when attempting to establish placement of an endotracheal tube: the fiberoptic bronchoscope is directed into the patient's trachea to act as a guide for the ETT which is passed over the fiberoptic bronchoscope to achieve proper placement of the ETT. In both “guide” situations, the obturator and the fiberoptic scope, hereinafter referred collectively as “guide(s),” have outer diameters that are less than the inner diameter of the ETT creating a gap between the guide and the ETT. Often times this gap creates difficulty for passing the ETT into the patients airway as the ETT “catches” or gets “hung up” on the laryngeal inlet because of the existing space between the guide and the ETT. Anatomically, as the endotracheal tube is slid over the guide, the ETT wants to move posteriorly but is brought anteriorly by the guide causing the gap (diameter difference between the guide and the ETT) existing between the guide and the ETT to be maximally exaggerated. Therefore, not only is the risk of the ETT getting caught on the laryngeal tissue increased but the tissue is now exposed to the hard bevelled edge of the plastic ETT increasing the likelihood of laryngeal injury (haematoma, vocal cord dysfunction, pain, arytenoid dislocation).

In the event that an ETT requires replacement, such as when an ETT is non-functional or a larger diameter ETT is needed for therapeutic intervention, an obturator is passed completely through the ETT and into the trachea of the patient. The existing inserted ETT is then removed leaving the obturator in place to act as a guide for the replacement ETT. Because the tissue around an existing ETT often becomes edematous and engorged the laryngeal inlet may collapse down on the obturator once the existing ETT is removed. This not only prevents the ability to oxygenate and ventilate the patient but very commonly prevents/increases the difficulty for passage of the replacement ETT into the trachea. In this event excessive force and rotation is usually required to overcome the “hang-up” of the ETT on the laryngeal anatomy. If these measures are unsuccessful the operator may need to downsize the ETT (i.e. decrease the diameter difference between the oburator and the ETT) to facilitate replacement. When this difficulty is encountered the patient is not able to be oxygenated or ventilated. In addition, the insertion of the replacement ETT with increased force tends to cause trauma or bleeding to the airway increasing the complications associated with ETT exchange. Several exchange obturators have been designed with a hollow lumen specifically for the purpose to allow passage of oxygen to the patient during the exchange time. Further, obturators have been designed with hollow lumens to allow the passage of oxygen into the patient's lungs while difficulty passing the ETT is being encountered. However, this is only a brief temporizing measure as the size of the lumens are extremely small and still do not circumvent the problem associated with the “hang-up” of the ETT.

In situations where placement of an ETT has not been established, a fiberoptic bronchoscope is commonly used when difficulty placing an endotracheal tube is anticipated or when neck immobility must be maintained (risk of cervical spinal injury with movement exists). Typically, the fiberoptic scope is placed through an ETT so that the forward end of the scope can be directed into the trachea by following the anatomy visualized on a viewing screen. The ETT placed over the scope is then slid or “railroaded” into the trachea. Often, because of the difference in diameters between the bronchoscope and the ETT, passage of the ETT is prevented as it gets “caught on” the anatomy at the laryngeal inlet. Again, excessive force and “corkscrewing” of the ETT is used to assist passage of the ETT into the airway. Because the patient is often times awake this is very disturbing as patients may become extremely combative and fearful increasing the degree of difficulty for placing the ETT and also increasing the risk of injury to both the laryngeal tissue and cervical spine (if immobility required). Furthermore, if passage of the ETT is not successful the scope needs to be withdrawn and an ETT of smaller diameter is placed on to the scope and placement in the same manner is reattempted.

Accordingly what is needed is a device and method for placing an ETT in a patient and a device and/or a method for replacing an ETT that has been placed in a patient that operates to facilitate placement of the ETT so that the ETT will not catch or get hung up on tissue during its placement.

### SUMMARY OF THE INVENTION

The above mentioned difficulties are overcome with the illustrative method and device for placing an ETT in a patient. The device includes an elongated tubular structure having an airway therein, which is insertable into the passageway of the ETT to allow rescue oxygenation and ventilation during placement of the ETT. Furthermore, the device includes a stylet that is adapted to slide smoothly over the tubular structure, such as by use of an activated lubricant and preferably the device includes means for which the stylet can slide on maintaining its orientation in relation to the tubular structure. Preferably the stylet has a tapered section with an expandable portion that gradually increases in diameter as it goes from distal to proximal on the stylet. When slid over the tubular structure the tapered portion of the stylet gently opens up the laryngeal tissue, preventing the ETT from catching on the tissue. The ETT then smoothly passes through the glottic opening and into the trachea without difficulty. As a result, this device for exchanging an established ETT and for primary placement of an ETT is a significant advancement in overcoming major obstacles and improving patient safety while placing an ETT.

Illustratively, the method for replacing a placed ETT tube includes placement of the hollow tubular structure through the ETT. The ETT is removed by being pulled over the tubular structure while it remains in the patient's airway. Prior to placement of the tubular structure the ETT and stylet have already been prepared to allow efficient replacement of the ETT. Preparation of the ETT occurs by initially, positioning the stylet into the ETT such that the diameter of the tapered portion of the stylet at the distal end of the ETT equals the outer diameter of the ETT. To facilitate passage of the stylet through the ETT the ETT and/or stylet preferably are lubricated. The ETT and stylet are then slid over the tubular structure. Preferably the device includes means for maintaining the orientation of the stylet and the ETT in relation to the tubular structure to assist in the smooth passage into the patient's airway. As the taper section of the stylet enters the laryngeal inlet the tapered shape gently opens the collapsed tissue allowing a smooth acceptance of the ETT into the patient's airway. Once the ETT is positioned correctly, the stylet and tubular structure are removed from the rearward end of the ETT while holding the ETT securely in place. Correct position of the ETT within the patient is then confirmed within the patient and the ETT is attached to the ventilator machine.

Illustratively, the method for placing an ETT includes using a tubular structure, such as a fiberoptic bronchoscope, and placing a stylet and endotracheal tube over the tubular structure. The stylet is placed into the lumen of the ETT in the same fashion as described above. In addition, the inner surface of the lumen and the outer surface of the tubular structure are preferably lubricated to provide smooth longitudinal movement as the stylet and the ETT are slid along the long axis of the tubular structure. When the laryngeal inlet is encountered the tapered design of the taper section of the stylet gently opens the tissue allowing a smooth acceptance of the ETT into the patient's airway. The tubular structure and the stylet are then removed. Because the stylet has a hollow lumen permitting oxygen and carbon dioxide exchange, the placement of the ETT can be confirmed before or after the stylet is removed. After correct placement of the ETT has been verified the ETT is then attached to the ventilating machine and secured.

A significant departure in the art includes the use of the stylet with a taper portion that can be slid over a tubular structure, such as an obturator or bronchoscope, for placement of an ETT. The tapered portion is formed such that it surrounds the distal end of the stylet and being substantially tapered such that the diameter of the tapered portion generally increases from its distal end to its proximal end. In one form of the invention the tapered portion of the stylet is expandable and includes a primary lumen; and the inflation means comprises a secondary lumen formed within the outer wall of the stylet offset from the primary lumen so as to communicate between the proximal end and the distal end of the stylet. The secondary lumen provides the communication between the expandable portion of the taper section and the inflation port so that air and possibly liquid (oxygen, air, medicated and non-medicated fluids will be referred to collectively as “fluid(s)”) can be passed to and from the expandable portion to inflate and deflate the expandable portion, respectively. In addition, the taper section acts to maintain the ETT there between. This creates a single-moving unit between the stylet sleeve and the ETT allowing easier, more controlled passage along the tubular structure, such as an obturator and fiberoptic bronchoscope.

### BRIEF DESCRIPTION OF THE DRAWINGS

To provide a more complete understanding of the present invention and further features and advantages thereof, reference is now made to the following description taken in conjunction with the accompanying drawings, in which:

FIG. 1 is a partial view showing the oral passageway of a patient for the exchange of gases between the lungs and the outside atmosphere and showing an exemplary embodiment of a conventional ETT placed in the air passage way of the patient and the tubular structure of the device of the present invention positioned within the ETT;

FIG. 2 is a partial view showing the oral passageway of a patient showing the ETT of FIG. 1 removed and the tubular structure in place in the air passage of the patient;

FIG. 3 is a perspective view of a preferred embodiment of the changing device of the present invention showing the stylet having a tapered section with an expandable portion and a proximal end cap;

FIG. 4 is an enlarged partial sectional view of the expandable portion of the tapered section of the stylet of FIG. 3 taken along line 4;

FIG. 5 is an enlarged sectional view of the stylet of FIG. 3 taken along line 5-5;

FIG. 6 is an enlarged partial perspective view of the stylet of FIG. 3 showing the end cap and inflation line assembly; and

FIG. 7 is an enlarged partial perspective view of the tapered portion of the stylet of FIG. 3, partially in section, showing the interior space formed between the tubular structure and the stylet.

### DETAILED DESCRIPTION OF THE INVENTION

The present invention relates to a device and method of performing endotracheal intubation and more particularly to a new and novel apparatus and method for placing an ETT or for replacing an ETT that has been placed in a patient. In describing the preferred embodiments of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose.

For purposes of the description of the present invention, the terms “forward” and “forwardly” are intended to refer to the direction towards the patient receiving the intubation device, whereas the terms “rear” and “rearwardly” are intended to refer to the direction away from the patient receiving the intubation device. The term “proximal” refers to a position away from the patient receiving the intubation device, whereas the term “distal” refers to a position towards the patient receiving the intubation device.

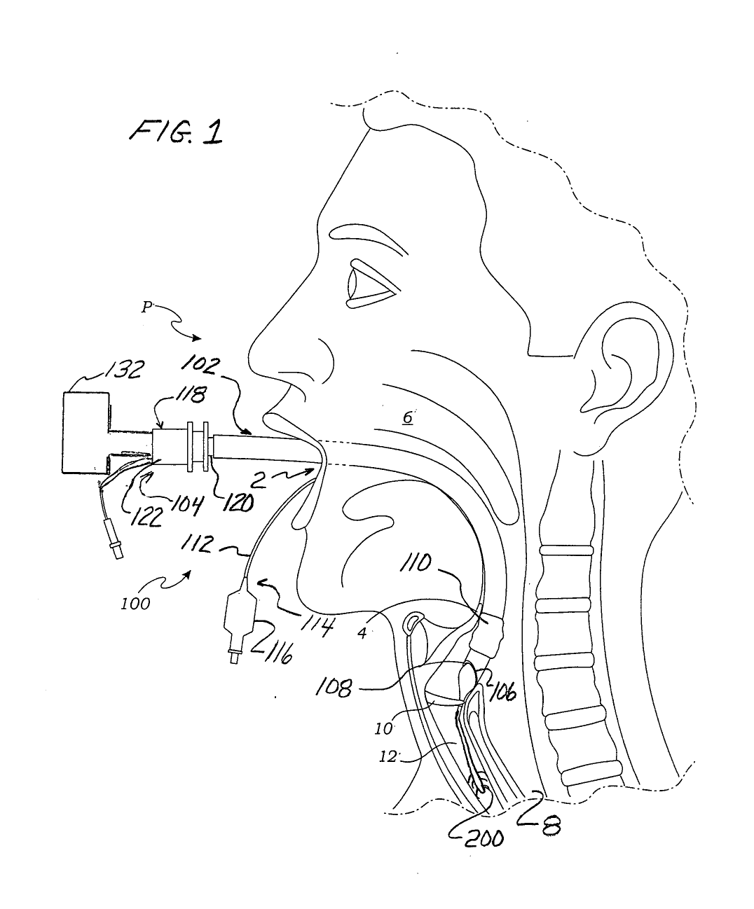

Referring to FIG. 1, a partial cross-section of a patient P is shown illustrating the mouth 2, the epiglottis 4, the nasopharynx 6, the esophagus 8 that operates to transfer food to the stomach (not shown), the larynx 10, and the trachea 12 that operates to provide a passageway for the exchange of gasses between the lungs (not shown), the alveoli (not shown), and the outside atmosphere A.

A conventional elongated ETT 102 having a proximal open end 104 and a distal open end 106 and a longitudinal bore 108 there between is shown placed in a patent P. The ETT 102 is formed of a pliable semi-rigid, soft plastic material such as, but not limited to, a polyethylene, a polypropylene, or like material. Preferably, the lower distal end of the ETT 102 includes one or more inflatable bladders or balloons 110 which is attached to a conventional air device (not shown) through an air line 112 such that when inflated the bladder 110 operates to prevent ventilation gas flowing through the ETT 102 from escaping outwardly from the trachea 12 of the patient P. A portion of the air line 112 is preferably positioned within the wall of the ETT 102 and provides flow communication between the bladder 110 and an external air source (not shown). The proximal end 114 of the air line 112 is provided with a flow valve 116 to permit a syringe (not shown) or other inflation device to be placed in flow communication with the air line 112 for injecting a predetermined amount of air into the bladder 110.

The proximal open end 104 of the ETT 102 is provided with a fitting 118 having a neck portion 120 for inserting longitudinally within the proximal open end 104 and an adaptor 122 for connecting the ETT 102 to a respirator or other ventilating apparatus or oxygen supply, anesthesia supply, or some other medical gas supply.

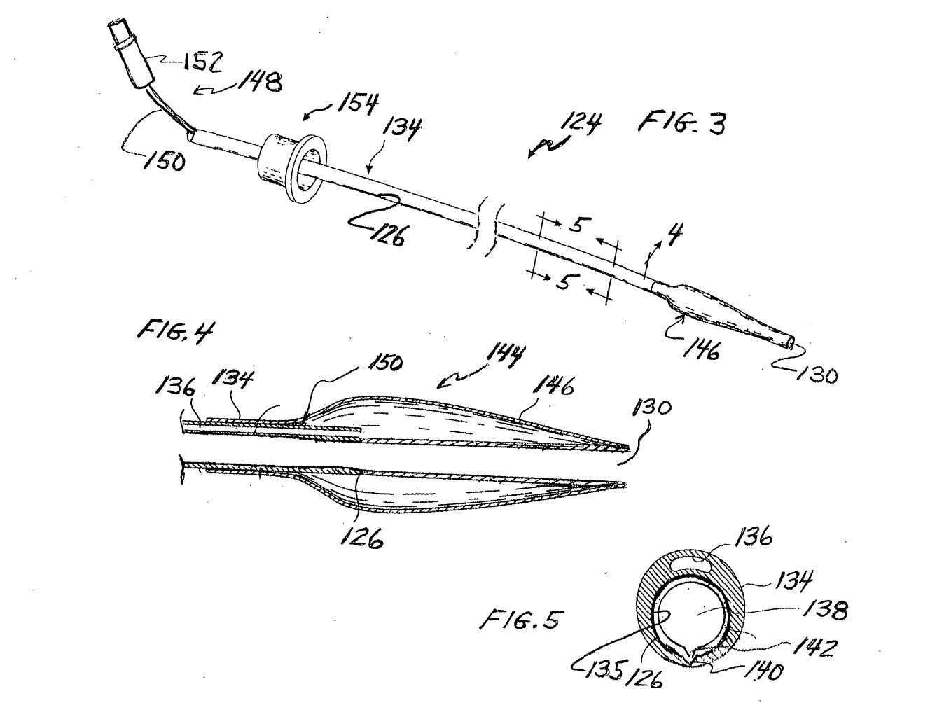

Referring to FIGS. 1 through 5, a device 124 for use in placing an ETT or for replacing a placed ETT 102, operates as a tubular obturator or as a fiberoptic bronchoscope, is positioned within the ETT 102. Preferably the device 124 comprises an elongated hollow tubular structure 126 having an open proximal end 128 and an open distal end 130 and is formed from a semi-flexible or flexible material that can bend easily to follow the path of the placed device 124. In a preferred embodiment of the invention the tubulae structure 126 is formed such that the distal portion of the tubular structure 126 is more flexible than the proximal portion of the tubular structure 126. Preferably, the tubular structure 126 has a very soft, pliable and rounded distal end 130 so that when placed into the patient P it does not cause or reduces the possibility of injury to the airway tissue. The open proximal end 128 is provided with a conventional removable fitting 132 for connecting to a respirator or other ventilating apparatus or oxygen supply, anesthesia supply, or some other medical gas supply (not shown).

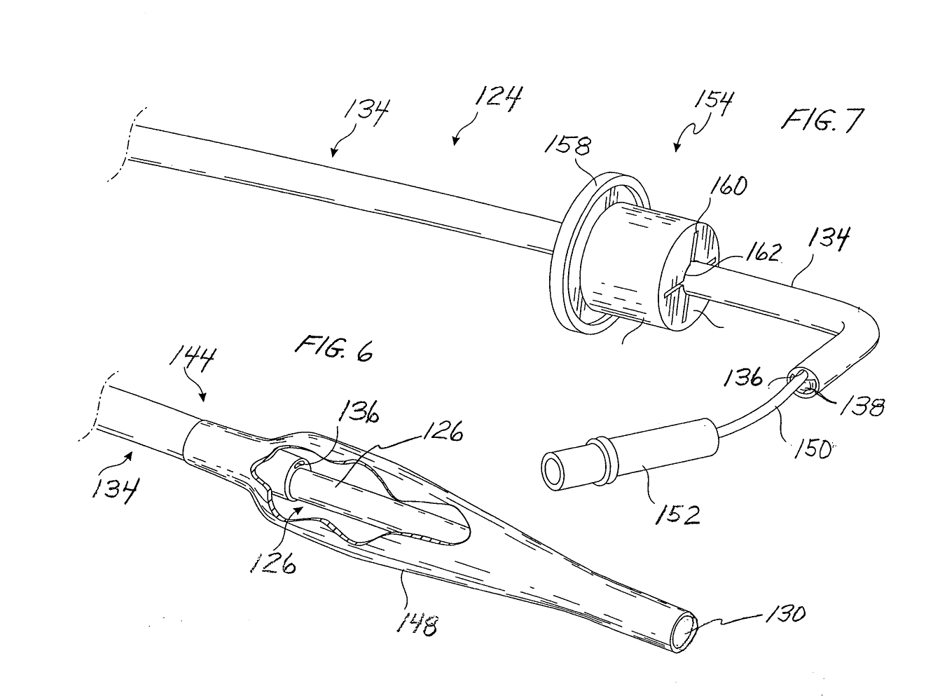

The device 124 further comprises a soft and pliable plastic stylet 134 formed from a biocompatible material such as a plastic, like a polyethylene, polypropylene, polyvinylchloride, or the like, or a rubber composition. The stylet 134 is adapted to slide longitudinally over and longitudinally along the tubular structure 126. As shown in FIGS. 5 and 6, the stylet 134 includes a lengthwise secondary lumen 136 and a primary lumen 138. The primary lumen 138 has a relatively circular cross-section into which the tubular structure 126 is inserted. Preferably the profile of the secondary lumen 136 is somewhat crescent shaped, however, it will be appreciated by those skilled in the art that such cross-section is merely exemplary and the invention is not so limited to such a cross-section. In the preferred embodiment the portion of the wall of the stylet 134 in which the secondary lumen 136 is formed is relatively thicker so as to accommodate this extrusion. It should now be understood that numerous other shapes, wall thicknesses, configurations and the like for the stylet 134 and its one or more lumens may be utilized without departing from the spirit and scope of the invention. As shown in FIG. 5, the inner wall 135 of the stylet 134 includes means for maintaining the orientation of the stylet 134 and the ETT 102 in relation to the tubular structure 126. In a preferred embodiment the means comprises a slot 140 that corresponds to and adapted to receive a projection or ridge 142 formed along the outer surface of the tubular structure 126. During operation, the stylet 134 is moved longitudinally forwardly such that the slot 140 rides along the ridge 142 of the tubular structure 126 thereby maintaining the orientation of the stylet 134 in relation to the tubular structure 126 and guiding the stylet 134 and the ETT 102 into the trachea 12.

Preferably, the distal ends of the ETT 102, the tubular structure 126, and the stylet 134 are rounded or tapered to minimize trauma to the tissue of the patient's airway during insertion. Referring more specifically to FIGS. 3, 4 and 6, the distal end of the stylet 134 is provided with a taper section 144. In a preferred embodiment the taper section includes an expandable portion 146 that operates to expand radially outwardly such as when air is directed downwardly through a relatively small inflation line assembly 148 comprising a relatively small diameter inflation line 150 that includes at its proximal end a one-way check-valve 152. As shown, the taper section 144 has a general cone shape and gradually increases in diameter from its distal to its proximal end. The inflation line 150 is installed in the proximal end of the secondary lumen 136 so as to be in flow communication therewith. The use of the one-way flow check-valve 152, air can be introduced through the valve 152 and into the secondary lumen 136 without escaping thereby inflating the expandable portion 146 of the taper section 144. It should be understood that the taper section 144 may not exist only as having an inflatable expandable portion that dilates with the input or release of air but that it may instead include a material that maintains the tapered form but can be retracted or pulled into the inner lumen of the ETT 102 or reduced in diameter when the stylet 134 is pulled rearwardly such that the taper section 144 is pulled into the ETT 102 thus allowing for easy removal. This material can be foam or gel or any other material that can be made into a tapered form that can fit different ETT 102 sizes and that allow for easy removal of the stylet 134 by being pulled through the distal end 106 of the ETT 102. In operation the taper section 144 is effective for reducing or eliminating the difference in diameters or gap between the distal end of the tubular structure 126 and the distal end of the ETT 102.

Referring more specifically to FIGS. 3 and 7, an end cap 154 is positioned on the stylet 134 substantially at its distal end. The end cap 154 is configured with an annular body 156 preferably sized and configured to meet ASTM/ISO 5356 standards for 15mm connectors used in conventional medical breathing circuits, equipment and devices. The distal end of the end cap 154 is formed with a radially-outwardly extending flange 158 to facilitate grasping the end cap 154 for engagement and disengagement with other connectors. The distal end of the end cap 154 is defined by a planar wall 160 forming the bottom surface of the inside, or female portion, of the connector and having an aperture 162 for the passage of the tubular structure 126 and stylet 134. Furthermore, the proximal planar wall 162 is formed with a plurality of radial slots 164 each intersecting the aperture 162 so as to effectively define the wall 166 as a plurality of living hinges each flexing axially substantially about the proximal perimeter of the annular body 156. While four slots 164 roughly ninety degrees (900) apart, and thus four living hinge sections that are roughly quadrants, are shown, it will be appreciated that virtually any number of slots 164 and resulting hinge sections may be formed within the planar wall 160 without departing from the spirit and scope of the invention. In more detail, the aperture 162 may be formed with an annular, distally projecting tubular body (not shown) substantially concentric therewith and having the same slots 164 passing there through, whereby these axially-extending annular wall portions cooperate with the hinge sections formed in the planar wall 160 to selectively flex in and out of contact with the stylet 134 during use, more about which is explained below. In an alternative embodiment, the aperture 162 itself is simply tapered from a smaller proximal diameter to a larger distal diameter. In any such configuration, it will be appreciated by those skilled in the art that as the tubular structure 126 and stylet 134 is passed through the aperture 162 of the end cap 154 and is advanced along the stylet 134 forwardly, the hinge sections of the planar wall 166 will flex distally, effectively opening up the aperture 162 and allowing the stylet 134 to slide there through. Then, if one were to attempt to retract the end cap 154 rearwardly along the stylet 134, the planar wall 166 would be forced back into a substantially planar configuration as the proximal perimeter of the aperture 162 effectively engages the outer surface of the stylet 134, thereby reducing the diameter of the aperture 162 and preventing travel of the end cap 154 along the stylet 134 in the rearward direction. This one-way movement of the end cap 154 along the stylet 134 resulting from the construction of the planar wall 156 with aperture 162 and slots 164 provides several functional advantages during use, as explained more below. Those skilled in the art will appreciate that numerous other one-way, or unidirectional, mechanical arrangements involving living hinges and the like, now known or later developed, may be employed in the end cap 154 of the present invention and that the exemplary embodiment of the end cap 154 shown and described is merely illustrative.

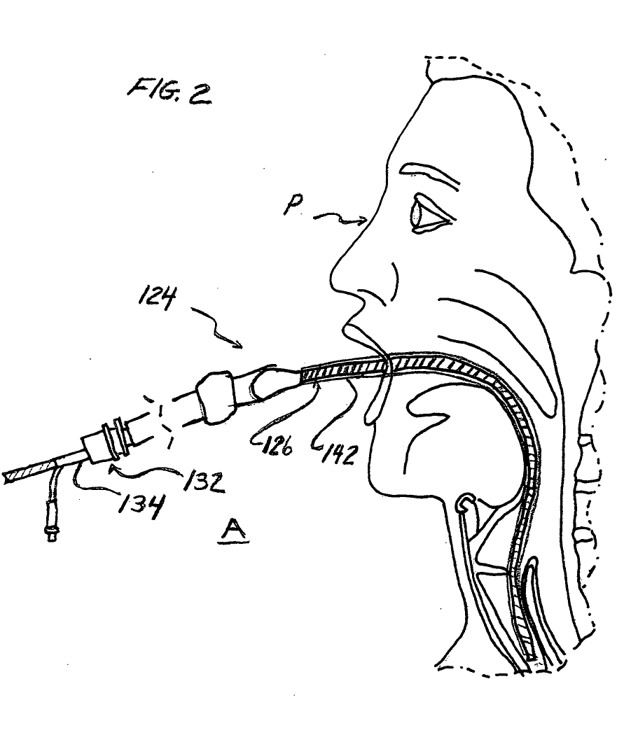

Referring to FIGS. 1, 2, and 6, in operation, an ETT 102 is positioned in the patient's air passage way and into the trachea 12. In cases requiring the ETT 102 to be replaced, the tubular structure 126 of the device 124 is inserted into the proximal end 104 of the ETT 102 and is longitudinally moved through the ETT 102 towards and out the distal open end 106 (as shown in FIG. 1) (step 1). Once the tubular structure 126 has been properly inserted such that the distal end 130 of the tubular structure 126 is positioned within the trachea 12. Ventilation can then be supplied through the tubular structure 126 to the patient P (step 2). The inserted ETT 102 can then be extracted from the patient P by simply sliding the ETT 102 longitudinally rearwardly along the tubular structure 126 (as shown in FIG. 2) leaving the tubular structure 126 in the patient's airway (step 3). Prior to removal of the ETT, the stylet 134 and the tubular structure 126 are prepared by lubricating the inner surface of the stylet 134 and the outer surface of the tubular structure 126 with a lubricant such as saline, water, silicone, or a water activated lubricant so the two surfaces easily pass over each other (step 4). The stylet 134 is then placed within the “new” ETT 102 (step 5) such that the universal connector (not shown) positioned on the ETT 102 is seated into the end cap 154 of the stylet 134. The end cap 154 can then be slid longitudinally along the stylet 134 until it's distal end is in the desired position in relation to the tapered section 144 of the stylet 134 (step 6). If the tapered section 144 exists having an expandable portion 146 the medical personnel inflates the expandable portion 146 of the tapered section 144 of the stylet 134 prior to starting the procedure.

The “old” ETT is then removed (step 7) by sliding the ETT rearwardly along the tubular structure 126 and the “new” or replacement ETT 102 is then inserted into the patient's mouth 2 (step 8) such that the “new” ETT 102 together with the stylet 134 are slid over the tubular structure 126 such that it is moved behind the epiglottis 4 above the esophagus 8 (FIG. 1) and towards the larynx 10 using the tubular structure 126 as a guide. In order to aide in insertion of the “new” ETT 102 and to avoid trauma to sensitive throat tissue, when the forward end of the ETT 102 approaches the larynx 10 (laryngeal inlet), the tapered section 144 of the stylet 134 moves into the larynx 10 facilitating entry of the ETT 102 by operating to dilate the larynx opening to the diameter of the ETT 102 facilitating passage of the ETT 102 and stylet 134 forwardly through the larynx 10 and into the trachea 12 (step 9) (FIG. 5). It should now be apparent that the tapered design of the taper section 144 operates to gently open the tissue allowing smooth less traumatic acceptance of the “new” ETT 102 into the patient's airway despite edematous, distorted or reactive (non-paralyzed) anatomy. It should also now be apparent to those skilled in the art that by reducing the risk of tissue trauma often encountered with insertion of an ETT 102 into a patient's trachea 12, tissue injury to the larynx 10 and specifically the vocal cords is reduced. Further, the taper design helps to prevent the catching of the ETT 102 on the laryngeal tissue 10 and facilitates a rapid return of adequate oxygenation and ventilation through the “new” ETT 102. After the ETT 102 has been properly inserted into the trachea 12, the expandable portion 142, if used, is deflated (or pushed into a reduced diameter) and the tubular structure 126 and stylet 134 are slid rearwardly out through the proximal end 104 of the ETT 102 (step 11). It should also now be understood that the use of a tubular structure 126 permits rescue oxygenation and ventilation during replacement of the ETT 102.

Once the “new” ETT 102 is in position within the trachea 12, air is injected, such as by a syringe, to inflate the bladder 110 (step 12). The inflated bladder 110 then operates to provide a seal to eliminate any gas that is being supplied by the ETT 102 from passing back out through the larynx 10. The ETT 102 can then be conventionally connected to a respirator or other ventilating apparatus or oxygen supply, an anesthesia supply, or some other medical fluid supply. After use, the ETT 102 can be conventionally removed by first releasing air from the bladder 110 and slowly withdrawing the ETT 102.

It should now be apparent that once the tubular structure 126 is inserted into the trachea 12, oxygen or some other fluid can be immediately supplied to the patient P. The ETT 102 and stylet 134 can then be slid over the tubular structure 126 and into the trachea 12. The tubular structure 126 and stylet 134 can then be removed. It should be apparent to those skilled in the art that the ability to inject oxygen or some other fluid through the tubular structure 126 can provide critically needed oxygen or other fluid to the patient during the changing or installation of the ETT 102. The elongated tubular structure 126 can then be connected using the fitting 118 to a respirator or other ventilating apparatus or oxygen supply, an anesthesia supply, or some other medical fluid supply.

In another preferred embodiment of the invention, the distal end of the tubular structure is provided with a plurality of side vents 200 (FIG. 1) for increasing the oxygen supply to the patient during replacement of an ETT 102 should the distal opening of the tubular structure becomes blocked such as by mucous or tissue. In another preferred embodiment of the invention, said taper section has a length of about one-half inch to about three inches.

In another preferred embodiment of the invention the tubular structure includes indicator means 170 (FIG. 7), such as markings positioned on the outer surface of the tubular structure 126 or some other effective means for indicating the position of the tubular structure 126 in relation to the inserted and/or new endotracheal tube 102.

It should be understood that as used herein the term “tubular structure” is not limited to hollow tubular structures, but may be a solid tubular shaped structure, or a solid tubular shaped structures with fluid passages therein. It should also now be apparent that the device of the present invention provides a tubular structure of sufficient length and flexibility to be guided into the trachea of the patient and a stylet effective for sliding over the tubular structure for providing increased rigidity and guidance to the flexible tubular structure.

It should also be understood that the stylet 134 can be used by itself with other conventional tubular structures. For example, the stylet 134 can be sized such that its inner wall diameter is just slightly larger that the outside surface of a conventional tubular structure, such as an obturator, so that it can slide along the surface of the conventional tubular structure as described hereinabove.

It should now be apparent to those skilled in the art that the device of the present application can be quickly and easily inserted into an inserted ETT to form an unobstructed conduit to a patient's trachea in which oxygen, medications, and other fluids can be passed during the changing of the ETT. It should also now be apparent to those skilled in the art that the device eliminates or reduces the problems typically associated with the changing of endotracheal tubes because of the relatively soft stylet having a taper section that reduces the likelihood of trauma to the sensitive throat tissue during insertion of the “new” ETT. In addition, the device of the present application allows for delicate control and sensitivity necessary to intubate a patient quickly with a minimum amount of trauma to sensitive tissue.

It should also now be apparent that the taper portion of the stylet of the present invention can be expanded to a desired size for permitting the device to be used with different sizes of ETTs.

Although the foregoing invention has been described in some detail for purposes of clarity of understandings, it will be apparent that certain changes and modifications may be practiced within the scope of the appended claims. Furthermore, it should be noted that there are alternative ways of implementing both the method and device for implementing the method of the present invention. Accordingly, the present embodiments and examples are to be considered as illustrative and not restrictive, and the invention is not to be limited to the details given herein, but may be modified within the scope and equivalents of the appended claims.

What is claimed is:

1. A device for use in inserting an endotracheal tube or for replacing an existing endotracheal tube that has been placed in the airway of a patient with a new endotracheal tube, the device comprising:
a tubular structure effective for being inserted into the endotracheal tube; and
a stylet adapted for sliding longitudinally along said tubular structure;
wherein said stylet includes a taper section effective for dilating the opening of the larynx in the airway of the patient during insertion.

2. The device of claim 1 wherein said taper section has a diameter effective for minimizing the lip formed between the wall of the endotracheal tube and said stylet.

3. The device of claim 1 wherein said tubular structure operates as an obturator.

4. The device of claim 1 wherein said tubular structure is a bronchoscope.

5. The device of claim 1 wherein said taper section comprises an expandable portion.

6. The device of claim 1 wherein said stylet includes a primary lumen for receiving said tubular structure and a secondary lumen for providing flow communication between a fluid supply and said expandable portion.

7. The device of claim 5 wherein said expandable portion is in flow communication with an inflation line and wherein said inflation line includes a one-way flow valve.

8. The device of claim 1 further comprising an end cap positioned along said stylet and effective for preventing said stylet from moving rearwardly in relation to the new endotracheal tube during insertion.

9. The device of claim 1 having means for maintaining the orientation of said stylet in relation to said tubular structure.

10. A device for use in inserting or replacing an inserted endotracheal tube that has been placed in the airway of a patient with a new endotracheal tube, the device comprising:
tubular structure insertable into a passageway of the endotracheal tube and having distal and proximal ends and a passageway extending therethrough; and
a stylet adapted to slide longitudinally along said tubular structure, wherein said stylet comprises a taper section effective for facilitating entry of the endotracheal tube into the airway of the patient;
wherein said tubular structure is connected to an air or oxygen supply and is effective for providing oxygen to the patient.

11. The device of claim 10 further comprising an end cap for connecting said tubular structure to a ventilator or fluid supply.

12. The device of claim 10 wherein said tubular structure includes indicator means positioned a predetermined distance from said distal end for indicating the position of said tubular structure in the endotracheal tube.

13. The device of claim 10 wherein said tubular structure includes side vents positioned about said distal end for increasing oxygen flow to the patient during insertion of the endotracheal tube.

14. The device of claim 10 having means for maintaining the orientation of said stylet in relation to said tubular structure.

15. A method for replacing an existing endotracheal tube that has been placed in the airway of a patient with a new endotracheal tube comprising the steps of:
inserting the tubular structure into the existing endotracheal tube and moving the tubular structure forward through the existing endotracheal tube until the tubular structure is positioned within the trachea of the patient;
removing the existing endotracheal tube by sliding the endotracheal tube rearwardly along the tubular structure;
inserting a stylet having a taper section into the new endotracheal tube and longitudinally over the tubular structure and moving the stylet forwardly such that the taper section extends outwardly from the distal end of the new endotracheal tube;
sliding the stylet and the new endotracheal tube longitudinally along the tubular structure such that the new endotracheal tube is in its proper position within the trachea; and
removing the stylet and tubular structure rearwardly out through the new endotracheal tube.

16. The method of claim 15 wherein the taper section is effective for facilitating entry of the new endotracheal tube through the opening of the larynx and into the trachea of the patient.

17. The method of claim 16 wherein the taper section has an expandable portion.

18. Method for inserting an endotracheal tube in a patient, the method comprising the steps of:
sliding a tubular member through the primary lumen of the stylet having a taper section;
inserting the stylet into the new endotracheal tube;
moving the stylet forwardly such that the taper section extends outwardly from the distal end of the new endotracheal tube;
sliding the tubular structure forwardly such that its distal end is proper placed within the trachea of the patient;
sliding the stylet and the new endotracheal tube longitudinally along the tubular structure such that the endotracheal tube is in its proper position within the trachea; and
removing the stylet and tubular structure rearwardly out through the endotracheal tube.

19. The method of claim 18 wherein the taper portion includes an expandable portion.

20. The device of claim 18 wherein the tubular structure is a bronchoscope.

21. The device of claim 18 wherein the tubular structure operates as an obturator.
